(19) United States
(12) Patent Application Publication
(10) Pub. No.: US 2009/0069437 A1
(43) Pub. Date: Mar. 12, 2009

(54) Compositions for Oral Transmucosal Delivery of Metformin

(76) Inventors: Anna E. Gluskin, Toronto (CA); Muhammad W. T. Qazi, Richmond Hill (CA)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/887,284

(22) PCT Filed: Mar. 30, 2006
(86) PCT No.: PCT/CA2006/000472

§ 371 Date: Mar. 24, 2008

Related U.S. Application Data
(60) Provisional application No. 60/666,475, filed on Mar. 30, 2005.

(51) Int. Cl. A61K 31/155; A61P 3/10

(52) U.S. Cl. 514/635

(57) ABSTRACT

The invention relates to oral transmucosal pharmaceutical compositions comprising metformin or a pharmaceutically acceptable salt thereof, methods of using the compositions to treat various conditions, including diabetes, methods of preparing the compositions, and preparations for use in making the compositions.

### BACKGROUND OF THE INVENTION

Metformin and pharmaceutically acceptable salts thereof (e.g. metformin hydrochloride, N,N-dimethylimidodicarbonimidic diamide hydrochloride) have been used to treat a number of conditions, including diabetes, pre-diabetes, polycystic ovary disease and obesity. Metformin's mechanisms of action include decreasing plasma glucose levels (particularly, postprandial glucose levels), decreasing hepatic glucose production, decreasing lipid levels, increasing sensitivity to insulin, and/or decreasing intestinal absorption. Furthermore, metformin acts without causing hypoglycemia.

Oral formulations (tablets) of metformin (e.g. GLUCOPHAGE, Bristol Meyers Squibb Co.) are currently in use. Administration of oral formulations of metformin may result in a number of side effects. Adverse events associated with oral formulations of metformin use are often gastrointestinal in nature (e.g. anorexia, nausea, bloating, vomiting and occasionally diarrhea, etc.). Furthermore, oral formulations of metformin may give rise to a bitter aftertaste, which may lead to loss of appetite. These side effects often result in the failure of patients to comply with taking the medication, i.e. “compliance issues”. Compliance issues are prevalent in individuals of all ages, including children, who typically do not want to take medicines that taste bad.

Therefore, there is an important need for formulations of metformin that, at least, mitigate one or more of these problems to help with compliance.

### SUMMARY OF THE INVENTION

In accordance with a first aspect, the invention provides an oral transmucosal metformin composition comprising a pharmaceutically acceptable carrier and an effective amount of a pharmaceutical agent consisting of metformin or a pharmaceutically acceptable salt thereof contained in said carrier, said carrier being capable of delivering a pharmaceutically effective amount of said pharmaceutical agent to an oral mucosal membrane for absorption.

In accordance with a second aspect, there is provided a process for making the oral transmucosal metformin composition comprising mixing an effective amount of a pharmaceutical agent consisting of metformin or a pharmaceutically acceptable salt thereof with an effective amount of at least one absorption enhancer chosen from an alkali metal alkyl sulfate, glycerin, a bile acid or bile salt, lecithin, hyaluronic acid, octylphenoxypolyethoxyethanol, glycolic acid, lactic acid, chamomile extract, cucumber extract, oleic acid, linolenic acid, borage oil, evening primrose oil, polyglycerin, lysine, polylysine, triolein, monoolein, monooleates, monolaurates, menthol, polidocanol alkyl ethers, chenodeoxycholate, deoxycholate and pharmaceutically acceptable salts and analogues thereof to form a preparation. The preparation is combined with a suitable pharmaceutically acceptable carrier to make the present composition.

In accordance with a further aspect, the invention provides a method of using, and a use of, the composition to treat various conditions chosen from diabetes, pre-diabetes, obesity and polycystic ovary syndrome. The present composition can be useful to decrease plasma glucose levels, decrease hepatic glucose production, decrease lipid levels, increase sensitivity to insulin, decrease intestinal absorption of glucose, decrease hypoglycemia and reduce appetite. The method involves administering to a subject a composition according to the first aspect in order to treat such conditions. The invention also provides a use of the composition in the manufacture of a medicament for treating the same conditions. The composition can be maintained in the mouth for at least 1, 20 or 30 minutes. As well, the composition can be maintained in the mouth from 1 to 30, 1 to 20 or 1 to 9 minutes.

In accordance with yet a further aspect, the invention provides a preparation for use in making a composition according to the first aspect. The preparation comprises a pharmaceutical agent consisting of metformin or a pharmaceutically acceptable salt thereof, and an effective amount of at least one absorption enhancer chosen from an alkali metal alkyl sulfate, glycerin, a bile acid or bile salt, lecithin, hyaluronic acid, octylphenoxypolyethoxyethanol, glycolic acid, lactic acid, chamomile extract, cucumber extract, oleic acid, linolenic acid, borage oil, evening primrose oil, polyglycerin, lysine, polylysine, triolein, monoolein, monooleates, monolaurates, menthol, polidocanol alkyl ethers, chenodeoxycholate, deoxycholate and pharmaceutically acceptable salts and analogues thereof, wherein the pharmaceutical agent is present in a concentration of from about 5 to 90, 10 to 80, 20 to 80 or 20 to 50 w/w %, and the total concentration of the absorption enhancers is less than about 30, 20, 10, 7, 5, 2, 1, 0.5, or 0.01 w/w % all based on the total weight of the preparation.

The present invention has a number of advantages. By by-passing the gastrointestinal (GI) tract, gastrointestinal complications and side effects of oral formulations of metformin and its salts can be avoided. In known formulations that are ingested, a higher amount of pharmaceutical agent is required per dose due to the problem of degradation in the GI tract. The present compositions which deliver the pharmaceutical agent through oral mucosal membranes can be formulated with less active ingredient. This leads to cost savings and helps to improve the taste profile.

### BRIEF DESCRIPTION OF THE DRAWINGS

The foregoing and other objects, features and advantages of the invention will be apparent from the following more particular description of preferred embodiments of the invention, and the accompanying drawings.

FIG. 1 is a graph showing metformin plasma concentrations (ng/ml) over time. Series 1 depicts the metformin plasma concentration in an individual given an 850 mg metformin hydrochloride tablet to ingest. Series 2 and 3 represent the metformin plasma concentrations in two individuals who chewed three chiclets of chewing gum, each containing 212.5 mg of metformin.

FIG. 2 is a graph showing the amount of metformin released from chewing gum compositions according to the present invention over time.

FIG. 3 is a graph comparing metformin plasma concentrations (ppm) over time in individuals given a 429 mg metformin tablet to ingest with individuals who chewed gum containing 429 mg of metformin.

### DETAILED DESCRIPTION OF THE INVENTION

A description of preferred embodiments of the invention follows.

### Pharmaceutical Compositions

In one embodiment, the invention is an oral transmucosal metformin composition comprising:

an effective amount of a pharmaceutical agent consisting of metformin or a pharmaceutically acceptable salt thereof,

an effective amount of at least one absorption enhancer chosen from an alkali metal alkyl sulfate, glycerin, a bile acid or bile salt, lecithin, hyaluronic acid, octylphenoxypolyethoxyethanol, glycolic acid, lactic acid, chamomile extract, cucumber extract, oleic acid, linolenic acid, borage oil, evening primrose oil, polyglycerin, lysine, polylysine, triolein, monoolein, monooleates, monolaurates, menthol, polidocanol alkyl ethers, chenodeoxycholate, deoxycholate and pharmaceutically acceptable salts and analogues thereof, and

a pharmaceutically acceptable carrier, said carrier being capable of delivering a pharmaceutically effective amount of said pharmaceutical agent to an oral mucosal membrane for absorption.

The pharmaceutically acceptable salt of metformin can be metformin hydrochloride.

In one embodiment, the pharmaceutical composition is in the form of chewing gum comprising metformin hydrochloride in a concentration of from about 10 to 50 w/w %, sodium lauryl sulfate in a concentration of from about 0.01 to 2 or 0.01 to 0.5 w/w %, sodium glycocholate in a concentration of from about 0.01 to 2 or 0.01 to 0.5 w/w %, glycerin in a concentration of from about 2 to 10 or 2 to 7 w/w %, and a chewing gum base in a concentration of from about 10 to 90, 30 to 75, or 60 to 75 w/w %, all based on the total weight of the composition. In another embodiment, the composition is in the form of a hard candy or lozenge.

In another embodiment, the invention is a process for making an oral transmucosal metformin composition comprising:

mixing (a) an effective amount of a pharmaceutical agent consisting of metformin or a pharmaceutically acceptable salt thereof with (b) an effective amount of at least one absorption enhancer chosen from an alkali metal alkyl sulfate, glycerin, a bile acid or bile salt, lecithin, hyaluronic acid, octylphenoxypolyethoxyethanol, glycolic acid, lactic acid, chamomile extract, cucumber extract, oleic acid, linolenic acid, borage oil, evening primrose oil, polyglycerin, lysine, polylysine, triolein, monoolein, monooleates, monolaurates, menthol, polidocanol alkyl ethers, chenodeoxycholate, deoxycholate and pharmaceutically acceptable salts and analogues thereof, to form a paste;

mixing the paste with a gum base; and

forming the resultant mixture into chewing gum tablets, capsules, caplets or chiclets.

Pharmaceutically acceptable salts and analogues of any of the disclosed absorption enhancers are also within the present scope as are mixtures or combinations of any of these compounds.

The absorption enhancers are those that facilitate delivery of the pharmaceutical agent across oral mucosal membranes. As used herein, “facilitate” refers to increasing the rate and/or amount of pharmaceutical agent delivered across an oral mucosal membrane (e.g. by at least about 5%, 10%, 20%, 25%, 30%, 35%, 40%, 50%, 60%, or 75%) compared to a pharmaceutical composition in which the absorption enhancer is absent.

Each absorption enhancer can be present in a concentration of up to about 30, 20, 15, 10, 5, 2, 1, 0.5, or 0.01 w/w % based on the total weight of the composition. The total amount of absorption enhancers is less than about 30, preferably less than about 20, and more preferably less than about 10 or 7 w/w % based on the total weight of the composition.

The absorption enhancers are micelle forming compounds which serve to encapsulate the pharmaceutical agent and facilitate its delivery across oral mucosal membranes when the composition is formed into a solution in the oral cavity.

As will be appreciated by those skilled in the art, a micelle is a colloidal aggregate of amphipathic molecules in which the polar hydrophilic portions of the molecule extend outwardly while the non-polar hydrophobic portions extend inwardly. It is believed that the presence of the micelles significantly aids in the absorption of the pharmaceutical agent both because of their enhanced absorption ability, and also because of their size. In addition, encapsulating pharmaceutical agents in micelles protects the agents from rapid degradation.

It will be understood that each micelle can contain the pharmaceutical agent and one or more absorption enhancers (i.e. micelle forming compounds). Preferably, at least two micelle forming compounds are used to form mixed micelles. As used herein the term “mixed micelles” refers to at least two different types of micelles each of which has been formed using a different micelle forming compound. For example, the present compositions can comprise a mix of at least two different types of micelles: micelles formed between the pharmaceutical agent and one of the micelle forming compounds (e.g. alkali metal alkyl sulfate), and micelles formed between the pharmaceutical agent and at least one additional micelle forming compound (e.g. sodium glycocholate). It will be understood that each individual micelle can be formed from more than one micelle-forming compound as well.

The size of the micelles is preferably greater than 6 microns but can be smaller, such as from about 1 to about 10 nanometers, or from about 1 to about 5 nanometers. The shape of the micelle can vary and can be, for example, prolate, oblate or spherical; spherical micelles are most typical. It is believed that the extremely small size of the micelles helps the encapsulated pharmaceutical agent penetrate efficiently through the oral mucosae. Thus, the present compositions offer increased bioavailability of active drug, particularly across oral mucosae, when compared with pharmaceutical preparations known in the art.

Any alkali metal alkyl sulfate can be used in the present compositions, provided compatibility problems do not arise. Preferably, the alkyl is a C8 to C22 alkyl, more preferably lauryl (C12). Any alkali metal can be utilized, with sodium being preferred. While the alkali metal alkyl sulfate is generally present in a concentration of up to about 30 w/w %, a concentration up to about 5 w/w % of the total composition is preferred. Even more preferred is a concentration of up to about 1, 0.5 or 0.01 w/w % of the total composition.

As used herein, the term “bile acid” includes, but is not limited to, cholic acid derivatives such as cholic, glycocholic, chenodeoxycholic, taurocholic, glycodeoxycholic and taurodeoxycholic acids. Any bile acids, or salt thereof, can be used in compositions of the present invention. Preferred is sodium glycocholate. Because the present invention uses relatively low concentrations of bile salts, problems of toxicity associated with the use of these salts is minimized, if not avoided.

The lecithin can be saturated or unsaturated, and is preferably chosen from phosphatidylcholine, phosphatidylserine, sphingomyelin, phosphatidylethanolamine, cephalin, and lysolecithin.

Preferred salts of hyaluronic acid are alkali metal hyaluronates, especially sodium hyaluronate, alkaline earth hyaluronates, and aluminum hyaluronate. When using hyaluronic acid or pharmaceutically acceptable salts thereof in the present compositions, a concentration of between about 1 and 5 w/w % of the total composition is preferred, more preferably between about 1.5 and 3.5 w/w %.

The composition can further comprise an isotonic agent in a concentration of up to about 30, 20, 15, 10 or 6 w/w % of the total composition. Suitable isotonic agents include, but are not limited to, saccharides such as sorbitol and mannitol, and polyhydric alcohols such as glycerin, polyglycerin, propylene glycol and the like, and dibasic sodium phosphate. Preferred is glycerin. The isotonic agent serves to keep the micelles in solution. Glycerin can function both as a micelle forming compound and an isotonic agent; when dibasic sodium phosphate is used it will also serve to inhibit bacterial growth.

Optionally, the pharmaceutical composition can comprise one or more additional therapeutic agents (e.g. sulfonureas). As used herein, the term “therapeutic agent” refers to an agent that ameliorates a disease or symptoms associated with a disease, including preventing or delaying the onset of the disease symptoms, and/or lessening their severity or frequency. In one embodiment, the therapeutic agent is used to treat diabetes, pre-diabetes, obesity or polycystic ovary syndrome.

An effective amount of the pharmaceutical agent should be included in the present composition. As used herein, the term “effective amount” refers to that amount of the pharmaceutical agent needed to bring about the desired result, such as obtaining the intended therapeutic treatment or prevention of a disorder in a patient, or regulating a physiological condition in a patient. Such an amount will therefore be understood as having a therapeutic and/or prophylactic effect in a patient. It will be appreciated that the effective amount will vary depending on the particular agent used, the parameters determined for the agent, the nature and severity of the disorder being treated, the patient being treated, and the characteristics of the carrier used.

An “effective amount” can also be the amount required such that peak metformin plasma concentrations are approximately equal to the peak metformin plasma concentrations in a subject administered an oral metformin hydrochloride tablet (for example, a metformin hydrochloride tablet containing about 50, 100, 250, 500, 750, 800 or 1000 mg of metformin hydrochloride). As used herein, “approximately equal” means that the peak plasma concentration of metformin after administration of the pharmaceutical composition of the invention (assessed using standard bioavailability measurements) is within 10% of the peak metformin plasma concentration after administration of an oral tablet formulation of metformin.

It will be understood that any decrease in plasma glucose levels, hepatic glucose production, lipid levels, intestinal absorption or weight loss can be therapeutic and/or prophylactic as can be any increase in sensitivity to insulin. The precise dosage level should be determined by the attending physician or other health care provider and will depend upon well-known factors, including the age, body weight, sex and general health of the individual, and the use (or not) of concomitant therapies. Of course, the skilled person will realize that divided and partial doses are also within the scope of the invention. The determination of what constitutes an effective amount is well within the skill of one practicing in the art.

Pharmaceutically effective doses may be extrapolated from dose-response curves derived from in vitro or animal model test systems. They may also be determined by measuring the bioavailability of known oral formulations of metformin hydrochloride. The pharmaceutical composition of the invention can then be formulated in a dose having a bioavailability that approximates the bioavailability of known oral formulations.

The amount of the pharmaceutical agent can be from about 50 to 850 milligrams.

Typically, the present compositions will contain about 50 to 500 milligrams per dose. Depending on the dosing regimen, each dose can contain 50, 112.5, 250 milligrams or 500 milligrams. It will be appreciated that the amount will vary depending on, amongst other things, the release characteristics of the carrier employed. The amount of active ingredient will be adjusted so that the amount of pharmaceutical agent released will have the intended therapeutic and/or prophylactic effect.

Each dose can contain from about 5 to 90, more preferably from about 10 to 80 w/w %, and even more preferably from about 20 to 80 or 20 to 50 w/w % of pharmaceutical agent based on the total weight of the composition, depending upon the amount of the carrier present.

The present compositions optionally contain a stabilizer and/or a preservative. Phenolic compounds are particularly suited for this purpose as they not only stabilize the composition, but they also protect against bacterial growth and help absorption of the composition. A phenolic compound will be understood as referring to a compound having one or more hydroxy groups attached directly to a benzene ring. Preferred phenolic compounds according to the present invention include phenol and methyl phenol (also known as m-cresol), and mixtures thereof.

The compositions of the present invention can further comprise one or more of the following: inorganic salts; antioxidants and protease inhibitors. The amount of any of these optional ingredients to use in the present compositions can be determined by one skilled in the art.

The inorganic salt or salts should be ones which can provide additional stimulation to release insulin. Non-limiting examples of inorganic salts are sodium, potassium, calcium and zinc salts, especially sodium chloride, potassium chloride, calcium chloride, zinc chloride and sodium bicarbonate.

The antioxidant is used to prevent degradation and oxidation of the pharmaceutically active ingredients. The antioxidant can be chosen from tocopherol, deteroxime mesylate, methyl paraben, ethyl paraben, ascorbic acid and mixtures thereof, as well as other antioxidants known in the pharmaceutical arts. A preferred antioxidant is tocopherol. The parabens will also provide preservation to the composition.

Protease inhibitors serve to inhibit degradation of the pharmaceutical agent by the action of proteolytic enzymes. When used, protease inhibitors are preferably in a concentration of between about 1 and 3 w/w % of the composition. Any material that can inhibit proteolytic activity can be used, absent compatibility problems. Examples include but are not limited to bacitracin and bacitracin derivatives such as bacitracin methylene disalicylates, soybean trypsin, and aprotinin. Bacitracin and its derivatives are preferably used in a concentration of between about 1.5 and 2 w/w % of the total composition, while soybean trypsin and aprotinin are preferably used in a concentration of between about 1 and 2 w/w % of the total composition.

It will be understood by those skilled in the art that colorants, flavoring agents and non-therapeutic amounts of other compounds may also be included in the composition. When menthol is used as one of the micelle-forming compounds, it will also impart flavor to the composition.

Flavoring agents can be essential oils, essences, extracts, powders, acids and other substances capable of affecting the taste profile. Flavors which can be used include, but are not limited to, coconut, coffee, cola, chocolate, vanilla, grape fruit, menthol, licorice, anise, apricot, caramel, honey, pineapple, strawberry, raspberry, tropical fruits, cherries, cinnamon, peppermint, wintergreen, spearmint, eucalyptus and mint flavors. In one embodiment, the flavors are chosen from menthol, caramel, coffee, and cola.

Colorants that can be used are of natural or synthetic origin and must be approvable for use in foods or medicines.

The carrier can be formulated into various shapes such as animal shapes or stars to appeal further to children.

The compositions of the present invention can be stored at room temperature or at cold temperature.

The pharmaceutical agent is to be administered through oral mucosal membranes or “oral mucosae”. These include membranes of the mouth, throat, larynx, and esophagus. Membranes of the mouth are preferred, in particular, the buccal and sublingual mucosa. The sublingual mucosa includes the membrane of the ventral surface of the tongue and the floor of the mouth, and the buccal mucosa is the lining of the cheeks. The sublingual and buccal mucosae are relatively permeable, allowing for the rapid absorption and acceptable bioavailability of many drugs. Further, the buccal and sublingual mucosae are convenient, non-evasive and easily accessible. In comparison to the GI tract and other organs, the buccal environment has lower enzymatic activity and a neutral pH that allows for a longer effective life of the drug in vivo.

The carrier is designed to release a sufficient amount of pharmaceutical agent and reside in the mouth for a sufficient period of time for absorption of the agent, so as to produce a therapeutic and/or prophylactic effect in a patient. For improved absorption, the carrier is preferably one that can be moved around the mouth so as to contact an increased surface area of the oral mucosal membranes. In preferred embodiments, the carrier is formulated as a masticatable candy (e.g. chewing gum or taffey) or as a hard candy or lozenge that can be chewed or sucked on for a sufficient period of time while the candy is moved over oral mucosal membranes. The amount of pharmaceutical agent released is over about 50, 60, 70, 80, or 90% during the period of time in which the carrier resides in the mouth. This period of time is from about 1 to 30, preferably from about 1 to 20, and more preferably from about 1 to 10 minutes. When released in the mouth and dissolved in saliva, the pharmaceutical agent will be present in micellar form as it will be encapsulated by the micellar forming absorption enhancers used herein. The person skilled in the art would readily understand how to make suitable carriers based on the teachings herein and common knowledge in the art.

### Process for Making the Composition

The present invention also provides a process for making the pharmaceutical composition of the present invention. The present compositions can be prepared by mixing an effective amount of a pharmaceutical agent consisting of metformin or a pharmaceutically acceptable salt thereof with an effective amount of at least one absorption enhancer chosen from an alkali metal alkyl sulfate, glycerin, a bile acid or bile salt, lecithin, hyaluronic acid, octylphenoxypolyethoxyethanol, glycolic acid, lactic acid, chamomile extract, cucumber extract, oleic acid, linolenic acid, borage oil, evening primrose oil, polyglycerin, lysine, polylysine, triolein, monoolein, monooleates, monolaurates, menthol, polidocanol alkyl ethers, chenodeoxycholate, deoxycholate, and pharmaceutically acceptable salts and analogues thereof to form a preparation to be combined with a pharmaceutically acceptable carrier.

The process can comprise the step of adding one or more ingredients chosen from isotonic agents, stabilizers, preservatives, antioxidants, protease inhibitors and inorganic salts.

The mixing can be effected by use of a high speed stirrer such as a KitchenAid brand professional HD Series Mixer for laboratory use or the like.

To make a chewing gum composition, the process can further comprise the steps of:

mixing the preparation with a gum base; and

forming the resulting mixture into chewing gum tablets, capsules, caplets or chiclets.

The specific amounts of ingredients can be determined by one skilled in the art based upon the general guidelines provided herein.

### Method of Treatment

The invention provides a method of treating conditions chosen from diabetes, pre-diabetes, obesity, and polycystic ovary syndrome, comprising administering to the subject a composition in accordance with the first aspect of the invention. The present compositions can be useful in decreasing the plasma glucose level in a subject (e.g. postprandial glucose level), decreasing hepatic glucose production, decreasing lipid levels, increasing sensitivity to insulin, decreasing intestinal absorption of glucose, decreasing hypoglycemia, decreasing body weight, and/or reducing appetite.

Where the composition is in the form of a masticatable candy or hard candy or lozenge, the method includes chewing and/or sucking the candy for a sufficient period of time for release and absorption of the pharmaceutical agent in micellar form, so as to produce a therapeutic and/or prophylactic effect in a patient.

The pharmaceutical composition can be provided in one dose (e.g. one piece or gum or candy) or may be provided in multiple doses which are administered serially. The frequency of administration and amount of metformin or its salt taken per dose will be determined by doctor's prescription based on the nature and severity of the condition to be treated and other factors, including without limitation, the sex, weight, health and age of the subject.

The method can also include the steps of administering one or more other therapeutic agents to treat diabetes, pre-diabetes, obesity, and/or polycystic ovary syndrome and/or produce weight loss. Therapeutic agents for the above conditions are known in the art, and the dosing of a combination therapy can be determined by a physician or health practitioner. For example, a pharmaceutical composition of the invention can be administered at the same time as the other therapeutic agent(s), or alternatively at different times of the day. In particular, the pharmaceutical composition of the invention can be administered in combination with insulin for treating diabetes. When the pharmaceutical compositions of the invention are used in combination with insulin, the amount of insulin required for control of diabetes can be decreased.

As used herein, “diabetes” or “diabetes mellitus” refers to a condition characterized by hyperglycemia. The hyperglycemia can be a result of absolute or relative impairment in insulin secretion and/or insulin action. Methods for detecting hyperglycemia are known in the art, and generally involve measuring plasma glucose levels. In asymptomatic patients, diabetes can be diagnosed when the diagnostic criterion for fasting hyperglycemia is met: a plasma (or serum) glucose level of >=140 mg/dL (>=7.77 mmol/L) (recommended by the National Diabetes Data Group (NDDG)) after overnight fast on two occasions in an adult or child; or when a subject has fasting plasma glucose levels of >126 mg/dL (>6.99 mmol/L) (recommended by the American Diabetes Association). Diabetes includes type 1 diabetes (insulin dependent diabetes mellitus), in which the subject produces little or no insulin, and type 2 diabetes (non-insulin dependent diabetes mellitus), in which hyperglycemia results from both an impaired insulin secretory response to glucose and/or decreased insulin effectiveness in stimulating glucose uptake by skeletal muscle and in restraining hepatic glucose production (insulin resistance).

As used herein, “pre-diabetes” (also referred to as impaired glucose tolerance, i.e. IGT) refers to a condition that occurs when a subject's post-prandial plasma glucose level is s are higher than normal but not high enough for a diagnosis of type 2 diabetes. Methods for measuring plasma glucose levels are known in the art.

As used herein, “polycystic ovary syndrome” or “hyperandrogenic chronic anovulation” is a condition that may cause amenorrhea, but is usually characterized by irregular menses, mild obesity, and hirsutism, typically beginning in the pubertal years and worsening with time. Most patients have abundant cervical mucus on examination and elevated free estrogens. Levels of most circulating androgens tend to be mildly elevated. The ovaries may be enlarged with smooth, thickened capsules or may be normal in size. Typically, the ovaries contain many 2- to 6-mm follicular cysts, and thecal hyperplasia surrounds the granulosa cells. Large cysts containing atretic cells may be present.

As used herein, “obesity” refers to having a body weight more than about 30% greater than ideal body weight, as determined by a medical professional, and/or having a body mass index greater than about 27 as determined by a medical professional.

The terms “therapeutic,” “treatment,” and “treat” and as used herein, refer to ameliorating a disease or symptoms associated with a disease, including preventing or delaying the onset of the disease symptoms, and/or lessening the severity or frequency of symptoms of the disease.

Symptoms of diabetes and pre-diabetes include, but are not limited to dyslipidemia, obesity, arterial hypertension, and microvascular and macrovascular complications, for example, atherosclerosis, retinopathies, nephropathies and neuropathies. Symptoms of obesity include, but are not limited to diabetes (e.g. type 2 diabetes), coronary artery disease, peripheral arterial occlusive disease, myocardial infarction, peripheral arterial occlusive disease, dyslipidemias (e.g. hyperlipidemia), stroke, chronic venous abnormalities, orthopedic problems, sleep apnea disorders, esophageal reflux disease, hypertension, arthritis, infertility, miscarriages and cancer (e.g. colorectal cancer, breast cancer).

### EXAMPLES

### Making of Paste Preparation

A preparation for use in making a composition according to the present invention was made as follows.

### Example 1

Powdered metformin hydrochloride (available from Spectrum Chemicals), powdered sodium glycocholate (available from NutriScience Innovations, LLC), and powdered sodium lauryl sulfate (available from Charles Tennant and Bioshop) were put through a 100 mesh screen and the particles that passed through the screen were used to make the preparation.

At room temperature and a relative humidity of from 25 to 65%, 123.51 grams of liquid glycerin (available from Canada Colors and Chemicals Ltd.) was poured slowly into a high speed mixing machine and stirred for approximately two to three minutes. To this was added 4.83 grams of the powdered sodium glycholate and the two ingredients were mixed for a further two to three minutes. 4.82 grams of the powdered sodium lauryl sulfate was then added and the mixture was mixed for two to three minutes more to produce an opaque solution. 1000 grams of the powdered metformin hydrochloride was then added with mixing continuing for another 15 to 20 minutes to form a homogeneous paste having a doughy texture.

The paste thus formed is according to the present invention and contained metformin hydrochloride in a concentration of 88.25 w/w %, glycerin in a concentration of 10.90 w/w %, sodium glycocholate in a concentration of 0.43 w/w % and sodium lauryl sulfate in a concentration of 0.43 w/w %, all based on the total weight of the paste preparation.

### Example 2

The protocol of Example 1 was repeated again with slightly differing amounts of the starting ingredients to produce a paste according to the present invention having metformin hydrochloride in a concentration of 76.98 w/w %, glycerin in a concentration of 22.28 w/w %, sodium glycocholate in a concentration of 0.37 w/w % and sodium lauryl sulfate in a concentration of 0.37 w/w %, all based on the total weight of the paste preparation.

In Examples 1 and 2, the amount of glycerin used to make the paste can be reduced so as to produce a paste having as little as 10 w/w % of glycerin based on the total weight of the paste. The paste preparation can be combined with a suitable pharmaceutically acceptable carrier to produce a composition according to the present invention.

### Preparation of Chewing Gum Composition

### Example 3

The paste according to Example 2 above was made into a chewing gum composition (chiclets) according to another aspect of the invention. Each chiclet contained metformin hydrochloride in a concentration of 212.5 w/w %, glycerin in a concentration of 6.15 w/w %, sodium glycocholate in a concentration of 0.10 w/w %, and sodium lauryl sulfate in a concentration of 0.10 w/w %, all based on the total weight of the chiclet composition. The balance of each chiclet consisted of gum base.

While the chewing gum of this example contained glycerin in a concentration of 6.15 w/w %, the starting amount of glycerin used to make the paste may be adjusted downwardly to produce a chewing gum having as little as 3 w/w % of glycerin based on the total weight of the gum.

### Example 4

The amount of ingredients used in Example 3 to make the paste were adjusted so as to produce a gum composition comprising 850 mg of metformin hydrochloride, 246 mg of glycerin, 4 mg of sodium glycocholate and 4 mg of sodium lauryl sulfate.

In both Examples 3 and 4, the chewing gum was prepared in accordance with a known method as follows. A matrix material consisting of elastomers, emulsifiers and waxes was ground and placed in a traditional chewing gum mixer. Additional ingredients (sweeteners, flavorings, and coloring agents) were then added to form a palatable gum base. The paste was then added to the gum base in a ratio of about 276 parts of paste to 1000 parts of gum base and all of the ingredients were mixed to form a homogenous chewing gum mass. The warm gum mass was then removed from the mixer and formed into chewing gum pieces using conventional systems and machines. The gum pieces were left to harden and coated with an optional dragée coating, which contained additional coloring and flavoring agents.

Chewing gum compositions according to the present invention may be made using other known methods such as those described in U.S. Pat. Nos. 5,487,902, 6,344,222, 6,432,383 and 5,470,566, the teachings of which are incorporated herein by reference.

### Preparation of Candies and Lozenges

For example, the paste can be formed into candies and lozenges using known methods such as those disclosed in U.S. Pat. No. 5,470,566, for example, the teachings of which are incorporated herein by reference.

It can also be used to make chewable capsules as described, for example, in U.S. Patent Application Publication No. U.S. 2003/0095925 A1, the teachings of which are incorporated herein by reference.

### Tests Involving the Administration of Metformin Gum

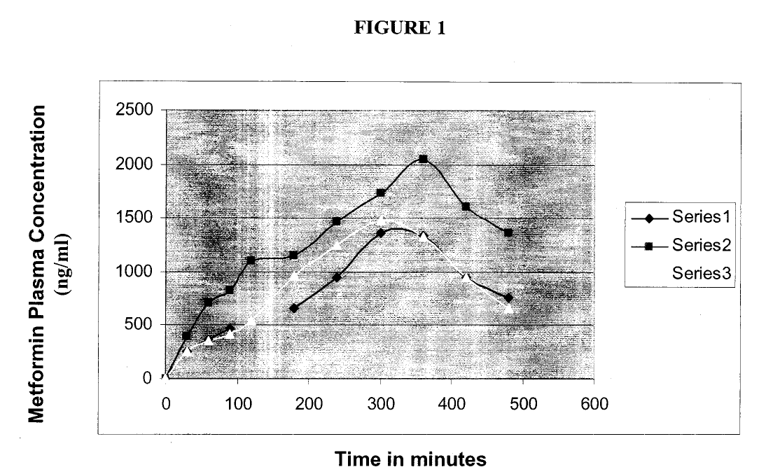

The chewing gum of Example 3 described above containing 212.5 mg of metformin hydrochloride per chiclet was administered to two subjects, individuals A and B. The subjects fasted overnight and the following morning were given one chiclet at time=0 which they chewed for 30 minutes and then spit out. After waiting 10 minutes, the subjects were given another chiclet to chew for another 30 minutes. The subjects then waited four hours and chewed a third chiclet before the first meal of the day for about 30 to 40 minutes. The concentration of metformin in the plasma of the individuals A and B (in ng/ml) was measured starting at time 0 and is plotted in FIG. 1. Series 2 represents the metformin plasma concentration for individual A. Series 3 represents the metformin plasma concentration for individual B.

A week before, individual B was given an 850 mg tablet of metformin hydrochloride sold in association with the trademark, GLUCOPHAGE, by Bristol-Meyers Squib Co. The tablet was ingested in the morning at time=0 after fasting overnight. The concentration of metformin in the plasma of individual B (in ng/ml) was measured starting at time 0 and plotted in FIG. 1 (see Series 1).

Peak plasma concentrations of metformin occurred after approximately 300 minutes in the control subject administered with GLUCOPHAGE and in one of the subjects administered with the metformin gum. Peak plasma concentration of metformin in the second subject administered with the metformin gum occurred at approximately 360 minutes. In one of the individuals given the gum, the peak plasma concentration was over 2000 ng/ml as compared to a peak of under 1500 ng/ml in the subject given the GLUCOPHAGE tablet. These results indicate that the gum is as or more effective than the tablets in delivering metformin hydrochloride to the bloodstream of human subjects, despite having far lower concentrations of metformin hydrochloride per three|-chiclet dose.

Release of Metformin Hydrochloride from Chewing Gum

The paste preparation of Example 2 was made into three different chewing gum compositions using the known method described above. Flavored gum bases were used. The serial numbers used to identify each gum base and the chewing gum composition produced using each base are identified below.
Chewing gum composition 5475-01-1 made using cola flavored gum base 25084;
Chewing gum composition 5475-04-1 made using caramel flavored gum base 25046; and
Chewing gum composition 5475-05-1 made using coffee-caramel flavored gum base 25046.

The chewing gum compositions were in the form of one gram chiclets. The chiclets contained about 210, 217 and 214 mg of metformin hydrochloride respectively. It should be understood that that the actual amount of metformin hydrochloride can vary in either direction by up to 5%.

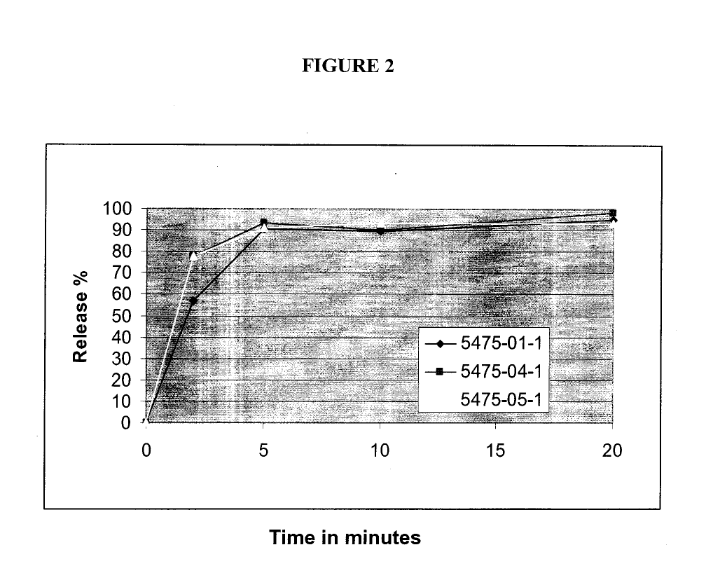

Each chiclet was put in a chewing machine containing a buffer solution. The chiclet was chewed at a rate of 60 chews per minute for 20 minutes. The amount of metformin hydrochloride released into the buffer solution was measured using high performance liquid chromatography (“HPLC”) at time=2, 5, 10 and 20 minutes as was the percentage of metformin hydrochloride released. The results are summarised in Table 1 below and the percentage of drug released was plotted (see FIG. 2).

TABLE 1

| | | Release % | | | |
|---|---|---|---|---|---|
| | Assay | Chewing time | | | |
| Sample | mg/g | 2 min. | 5 min. | 10 min. | 20 min. |
| 5475-01-1 | 210 | 57 | 91 | 89 | 95 |
| 5475-04-1 | 217 | 78 | 93 | 90 | 98 |
| 5475-05-1 | 224 | 78 | 91 | 93 | 94 |

Analytical data material to be found from ad hoc 9/04.43.06.

The initial release rate for gum composition 5475-01-1 is slower than for the other compositions due to the use of a slower release gum base.

The overall results show that metformin hydrochloride release is quite fast in all the compositions, with at least 90% of the pharmaceutical agent being released after just five minutes of chewing.

### Example 5

### Tests Comparing Metformin Gum with Metformin Tablets at the Same Dose Level

Chewing gum having the same composition as the composition of Example 3, except that the amount of metformin hydrochloride is 214.5 mg per chiclet (as opposed to 212.5 mg) was administered to a group often healthy volunteers (6 males, 4 females), with a mean age of 30 and 29.8 years respectively and a mean BMI of 23.9 and 21.49 respectively. At time=0, each subject was given two chiclets to chew for a total dose of 429 mg metformin hydrochloride.

As a control, |on another day|, the same group of subjects was each given one 429 mg tablet of metformin hydrochloride sold in association with the trademark, GLUCOPHAGE, by Bristol-Meyers Squib Co.

Plasma samples (300 microlitres) obtained from the subjects were submitted to solid phase extraction (spe) with weak cation exchange prior to analysis with HPLC.

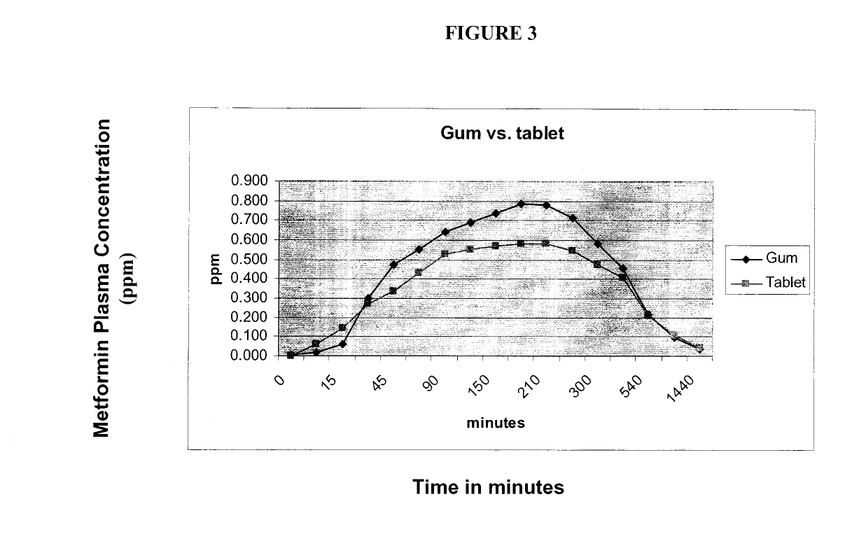

The concentration of metformin in the plasma of the subjects (in ppm) was measured starting at time=0 minutes, and additional sampling occurred at 5, 15, 30, 45, 60, 90, 120, 150,|180, 210, 240, 300, 360, 540, 720, 1440 minutes (24 hours). The volumes were plotted in FIG. 3.

A Corresponding Areas under the Curve (AUC) analysis by ANOVA (f-test) and a pairwise t-test indicated that there was no significant difference in plasma concentrations of subjects given the gum as compared to subjects given the GLUCOPHAGE tablet, although the plasma concentrations with the gum tended to be higher. Peak plasma concentrations of metformin occurred at approximately 200 minutes in both groups of subjects, with the peak plasma concentration almost 0.8 ppm in subjects given the gum versus almost 0.6 ppm in subjects given the GLUCOPHAGE tablet.

These results indicate that the gum is at least as effective as the tablets in delivering metformin hydrochloride to the bloodstream of human subjects.

While this invention has been particularly shown and described with references to preferred embodiments thereof, it will be understood by those skilled in the art that various changes in form and details may be made therein without departing from the scope of the invention encompassed by the appended claims.

1-36. (canceled)

37. A preparation for use in making an oral transmucosal metformin composition, the preparation comprising:
an effective amount of a pharmaceutical agent consisting of metformin or a pharmaceutically acceptable salt thereof; and
at least one absorption enhancer chosen from an alkali metal alkyl sulfate, glycerin, a bile acid or bile salt, lecithin, hyaluronic acid, octylphenoxypolyethoxyethanol, glycolic acid, lactic acid, chamomile extract, cucumber extract, oleic acid, linolenic acid, borage oil, evening primrose oil, polyglycerin, lysine, polylysine, triolein, monoolein, monooleates, monolaurates, menthol, polidocanol alkyl ethers, chenodeoxycholate, deoxycholate and pharmaceutically acceptable salts and analogues thereof;
wherein the pharmaceutical agent is present in a concentration of from about 5 to 90 w/w % based on the total weight of the preparation.

38. An oral transmucosal metformin composition comprising:
an effective amount of a pharmaceutical agent consisting of metformin or a pharmaceutically acceptable salt thereof;
at least one absorption enhancer chosen from an alkali metal alkyl sulfate, glycerin, a bile acid or bile salt, lecithin, hyaluronic acid, octylphenoxypolyethoxyethanol, glycolic acid, lactic acid, chamomile extract, cucumber extract, oleic acid, linolenic acid, borage oil, evening primrose oil, polyglycerin, lysine, polylysine, triolein, monoolein, monooleates, monolaurates, menthol, polidocanol alkyl ethers, chenodeoxycholate, deoxycholate and pharmaceutically acceptable salts and analogues thereof, wherein said at least one absorption enhancer is a micelle forming compound capable of forming micelles to encapsulate the pharmaceutical agent when the composition is formed into a solution in the oral cavity; and
a pharmaceutically acceptable carrier, said carrier being capable of delivering a pharmaceutically effective amount of said pharmaceutical agent to an oral mucosal membrane for absorption.

39. The composition of claim 38, including more than one absorption enhancer, wherein the total concentration of the absorption enhancers is less than about 30 w/w % of the total composition.

40. The composition of claim 39, wherein each absorption enhancer is present in a concentration of less than about 7 w/w % of the total composition.

41. The composition of claim 39, wherein one of the absorption enhancers is sodium lauryl sulfate.

42. The composition of claim 39, wherein one of the absorption enhancers is glycerin.

43. The composition of claim 39, wherein one of the absorption enhancers is sodium glycocholate.

44. The composition of claim 39, wherein the pharmaceutical agent comprises metformin hydrochloride.

45. The composition of claim 39, in the form of chewing gum comprising metformin hydrochloride in a concentration of from about 10 to 50 w/w %, sodium lauryl sulfate in a concentration from about 0.01 to 2 w/w %, sodium glycocholate in a concentration of from about 0.01 to 2 w/w %, glycerin in a concentration of from about 2 to 10 w/w %, and a chewing gum base in a concentration of from about 10 to 90 w/w %, all based on the total weight of the composition.

46. The composition of claim 39, wherein the pharmaceutically acceptable carrier is a hard candy or lozenge.

47. The composition of claim 39, wherein the oral mucosal membrane is the buccal membrane.

48. A process for making an oral transmucosal metformin composition comprising:
mixing (a) an effective amount of a pharmaceutical agent consisting of metformin or a pharmaceutically acceptable salt thereof with (b) at least one absorption enhancer chosen from an alkali metal alkyl sulfate, glycerin, a bile acid or bile salt, lecithin, hyaluronic acid, octylphenoxypolyethoxyethanol, glycolic acid, lactic acid, chamomile extract, cucumber extract, oleic acid, linolenic acid, borage oil, evening primrose oil, polyglycerin, lysine, polylysine, triolein, monoolein, monooleates, monolaurates, menthol, polidocanol alkyl ethers, chenodeoxycholate, deoxycholate and pharmaceutically acceptable salts and analogues thereof, to form a paste;
mixing the paste with a gum base; and
forming the resultant mixture into chewing gum tablets, capsules, caplets or chiclets.

49. A method of treating a condition chosen from diabetes, pre-diabetes, obesity and polycystic ovary syndrome, comprising administering to a subject having one of the afore-mentioned conditions an oral transmucosal metformin composition comprising:
an effective amount of a pharmaceutical agent consisting of metformin or a pharmaceutically acceptable salt thereof;
at least one absorption enhancer chosen from an alkali metal alkyl sulfate, glycerin, a bile acid or bile salt, lecithin, hyaluronic acid, octylphenoxypolyethoxyethanol, glycolic acid, lactic acid, chamomile extract, cucumber extract, oleic acid, linolenic acid, borage oil, evening primrose oil, polyglycerin, lysine, polylysine, triolein, monoolein, monooleates, monolaurates, menthol, polidocanol alkyl ethers, chenodeoxycholate, deoxycholate and pharmaceutically acceptable salts and analogues thereof, wherein said at least one absorption enhancer is a micelle forming compound capable of forming micelles to encapsulate the pharmaceutical agent when the composition is formed into a solution in the oral cavity; and
a pharmaceutically acceptable carrier, said carrier being capable of delivering a pharmaceutically effective amount of said pharmaceutical agent to an oral mucosal membrane for absorption.

50. The method of claim 49, wherein the composition is in the form of chewing gum, comprising metformin hydrochloride in a concentration of from about 10 to 50 w/w %, sodium lauryl sulfate in a concentration from about 0.01 to 2 w/w %, sodium glycocholate in a concentration of from about 0.01 to 2 w/w %, glycerin in a concentration of from about 2 to 10 w/w %, and a chewing gum base in a concentration of from about 10 to 90 w/w %, all based on the total weight of the composition.

51. The method of claim 50, wherein the condition is diabetes.

52. The method of claim 50, wherein the condition is obesity.

53. The method of claim 50, wherein the condition is polycystic ovary syndrome.

54. A method of decreasing the plasma glucose level, decreasing hepatic glucose production, decreasing lipid levels, increasing sensitivity to insulin, decreasing the intestinal absorption of glucose, decreasing hypoglycemia, decreasing body weight or reducing appetite comprising administering to a subject in need thereof an oral transmucosal metformin composition comprising:
an effective amount of a pharmaceutical agent consisting of metformin or a pharmaceutically acceptable salt thereof;
at least one absorption enhancer chosen from an alkali metal alkyl sulfate, glycerin, a bile acid or bile salt, lecithin, hyaluronic acid, octylphenoxypolyethoxyethanol, glycolic acid, lactic acid, chamomile extract, cucumber extract, oleic acid, linolenic acid, borage oil, evening primrose oil, polyglycerin, lysine, polylysine, triolein, monoolein, monooleates, monolaurates, menthol, polidocanol alkyl ethers, chenodeoxycholate, deoxycholate and pharmaceutically acceptable salts and analogues thereof, wherein said at least one absorption enhancer is a micelle forming compound capable of forming micelles to encapsulate the pharmaceutical agent when the composition is formed into a solution in the oral cavity; and
a pharmaceutically acceptable carrier, said carrier being capable of delivering a pharmaceutically effective amount of said pharmaceutical agent to an oral mucosal membrane for absorption.

55. The method of claim 54, wherein the composition is in the form of chewing gum, comprising metformin hydrochloride in a concentration of from about 10 to 50 w/w %, sodium lauryl sulfate in a concentration from about 0.01 to 2 w/w %, sodium glycocholate in a concentration of from about 0.01 to 2 w/w %, glycerin in a concentration of from about 2 to 10 w/w %, and a chewing gum base in a concentration of from about 10 to 90 w/w %, all based on the total weight of the composition.

56. The method of claim 55, wherein the method increases the subject's sensitivity to insulin.

57. The method of claim 55, wherein the method decreases the subject's intestinal absorption of glucose.

58. The method of claim 55, wherein the method decreases the subject's hypoglycemia.

59. The method of claim 55, wherein the method decreases the subject's body weight.

60. The method of claim 55, wherein the method decreases the subject's appetite.
